(19) United States
(12) Patent Application Publication
(10) Pub. No.: US 2025/0221964 A1
(43) Pub. Date: Jul. 10, 2025

(54) Substituted Isoxazoles as Selective Nav1.7 Inhibitors for Pain Treatment and Method of Pain Treatment

(71) Applicant: Board of Regents, The University of Texas System, Austin, TX (US)

(72) Inventors: DOUG FRANZ, Fair Oaks, TX (US); ROBYNNE NEFF, San Antonio, TX (US); SARA DIBRELL, Pasadena, CA (US)

(21) Appl. No.: 17/911,336

(22) PCT Filed: Mar. 12, 2021
(86) PCT No.: PCT/US21/22187

§ 371 Date: Feb. 10, 2023

(51) Int. Cl. A61K 31/42; C07D 261/18

(52) U.S. Cl. CPC A61K 31/42; C07D 261/18

(57) ABSTRACT

Tri-substituted isoxazole compounds and compound synthesis is disclosed. Tri-substituted isoxazole compounds having sodium gate channel Nav7 selectivity that are tunable for selectivity over Nav1.5. In particular, structure-activity relationship (SAR) studies demonstrated that subtype selectivity (Nav1.7 vs. Nav1.5) is improved with methylation of the amide nitrogen or ortho-substitution on the phenyl ring in the 5-position.

### BACKGROUND OF THE INVENTION

The annual cost of chronic pain has been estimated as being as high as $635 billion, which is greater than the combined yearly costs for cancer, heart disease and cancer. According to the Medical Expenditure Panel Survey in 2008, about 100 million adults in the United States were affected by chronic pain. Chronic pain limits suffers functional status and adversely impacts their quality of life. Pain also complicates medical care for other ailments.

To further complicate the problem, from 1999-2017 almost 400,000 people have died from opioid overdose, including both prescription and illicit opioids. According to the Centers for Disease Control and Prevention “prescription opioids can be used to treat moderate-to-severe pain and are often prescribed following surgery or injury, or for health conditions such as cancer. In recent years, there has been a dramatic increase in the acceptance and use of prescription opioids for the treatment of chronic, non-cancer pain, such as back pain or osteoarthritis, despite serious risks and the lack of evidence about their long-term effectiveness.” More than 191 million opioid prescriptions were dispensed to patients in the United States in 2017. Opioids are addictive and patients taking them develop tolerance to their pharmacologic benefits, necessitating increased dosages. Moreover, opioid administration frequently carries with it deleterious side effects which also limits their long-term use and effectiveness. Unfortunately, chronic pain continues to expand as an overwhelming burden to the health care system while current therapies suffer from limitations in efficacy, undesirable side effects and contribute to a growing drug addiction crisis in the U.S.

Accordingly, identifying alternatives for chronic pain treatment has become a high priority in the pain therapy and management field. Presented in this application are substituted isoxazole compounds having antagonistic activity at the Nav1.7 voltage-gated sodium channel.

Recent studies have suggested that most chronic pain states are maintained by persistent peripheral nociceptive triggers. As a result, the voltage-gated sodium channel Nav1.7 has attracted the greatest attention as an alternative pharmacological target due to strong genetic validation implicating its pivotal role in pain perception in the peripheral nervous system. Voltage-gated sodium channels are essential for electrogenesis in nerve cells. Genetic, structural and functional studies have shown that Nav1.7 regulates sensory neuron excitability and is a major contributor to several sensory modalities including human pain disorders. Other indications include sense of smell, cough reflex, and epilepsy. The role of Nav1.7 in acquired and inherited pain conditions, including chronic pain, has been well established. Small molecules that act as antagonists (synonymously, inhibitors) of Nav1.7 have been shown to be potential therapeutic agents for the treatment of chronic pain in humans.

Despite intense effort, however, small molecule inhibitors of Nav1.7 have yet to recapitulate the powerful analgesic phenotype observed in Nav1.7-null subjects shedding doubt on the druggability of this target. This lack of efficacy has, in part, been attributed to poor isoform selectivity of all reported inhibitors over other sodium channel subtypes, which elicits dose-limiting side effects. In particular, tuning out inhibition of the cardiac Nav1.5 subtype, which could lead to significant cardiovascular adverse events, has been a significant challenge to overcome.

### SUMMARY OF THE INVENTION

Substituted isoxazoles are potent inhibitors of Nav1.7 and their structure-activity relationships have been found tunable for selectivity over Nav1.5. Structure-activity relationship (SAR) studies demonstrated that subtype selectivity (Nav1.7 vs. Nav1.5) could be improved with methylation of the amide nitrogen or ortho-substitution on the phenyl ring in the 5-position.

It has been found that subtle structural differences have a profound influence on both IC50 potency and selectivity of Nav1.7 over Nav1.5.

It has been found that a 4-phenyl substitution increases Nav1.7 inhibition, particularly, where the 4-phenyl substituent is substantially orthogonal to the isoxazole ring.

The structural activity relationships between different 3,4,5 substitutions on the isoxazole ring have revealed that atropisomerism around the carbon-carbon bond at the 4-position affords chirality that enhances Nav1.7 inhibition and selectivity over Nav1.5.

A synthesis of atropisomeric 3, 4, 5-trisubstituted isoxazole is also disclosed.

### BRIEF DESCRIPTION OF THE DRAWINGS

FIG. 1 illustrates a restricted rotation axis of chirality of telenzepine as is known in the art.

FIG. 2 illustrates the core structure of the inventive tri-substituted isoxazole compound in accordance with the present disclosure.

FIG. 3 is a graph of the results from a mouse automated formalin test an isoxazole compound in accordance with the present disclosure.

FIG. 4 is a density function theory (B3LYP/6-31G) geometry minimization of a simplified methyl ester analog of compound in accordance with the present invention.

FIG. 5 is a pharmacophore model and SAR on the tri-substituted isoxazole Nav1.7 inhibitors in accordance with the present disclosure.

FIG. 6 is a synthetic pathway for atropisomeric 3, 4, 5 tri-substituted isoxazoles employed for SAR analysis in Nav1.7/Nav1.5 in vitro assays in accordance with the present disclosure.

### DETAILED DESCRIPTION OF THE PREFERRED EMBODIMENTS

A new class of isoxazole-based Nav1.7 inhibitors are disclosed that demonstrate potent inhibition of hNav1.7, tunable selectivity over hNav1.5 and possess ideal starting physiochemical properties for further drug development. As part of the structure-activity relationship (SAR) identification, a previously unrecognized structural feature in this class of compounds, atropisomerism, has been identified. The dramatic role that chirality and three-dimensionality plays in influencing biological activity is well established. However, atropisomerism, an example of axial chirality, has never been exploited in SAR studies of isoxazole-based small molecules despite their role as a privileged scaffold in medicinal chemistry. This has led to identifying a potent Nav1.7 inhibitor with high levels (>100×) of selectivity over Nav1.5 that can serve as a tool compound to further elucidate isoform selectivity and provide the foundation for a drug discovery program for chronic pain treatment. Without intending to be bound to theory, we posit that this previously unknown atropisomerism will serve as a fine-tuning mechanism to not only increase potency but also function as a unique architectural handle to enhance Nav1.7 selectivity over Nav1.5.

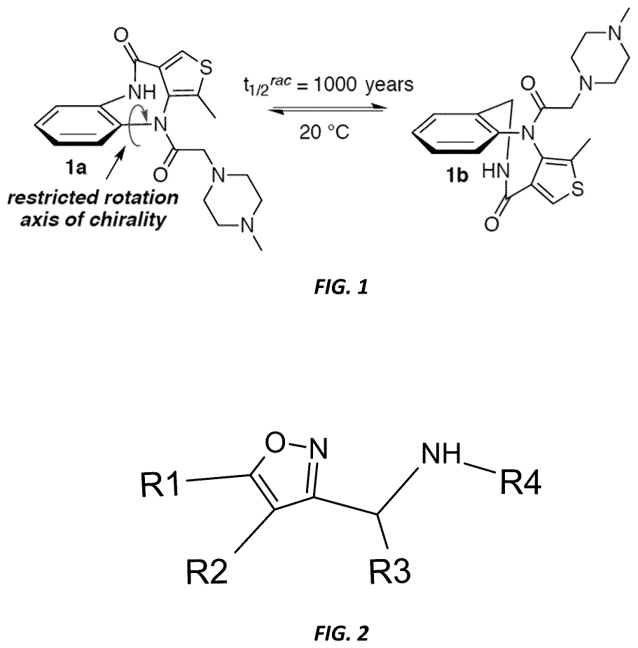

Drug discovery is intensely affected by chirality. For the past 20 years, medicinal chemists have increasingly tried to mimic nature's exquisite control of chirality into their synthetic drug discovery programs. The advantages of developing a single stereoisomer drug substance over mixtures (i.e., single enantiomer vs.racemate) are numerous and include improved efficacy, reduced off-target effects, and avoid stereospecific differences in pharmacokinetics and pharmacodynamics. However, an often-overlooked form of chirality in drug discovery is atropisomerism occurs due to a significant rotational barrier (˜22 kcal/mol) about a rotationally stable bond axis that imparts asymmetry into the molecule and allows for the potential isolation of conformational stereoisomers. As with classical sources of chirality, these conformationally distinct stereoisomers can have significant differential biological profiles. A well-known industrial example of differential activity due to atropisomerism is telenzepine, shown in FIG. 1. Telenzipine is a selective muscarinic antagonist for the treatment of peptic ulcers. The (−)-isomer was eventually found to be 500× less active with much less selectivity than the (+)-isomer at muscarinic receptors in the rat cerebral cortex. Unfortunately, strategies to address atropisomerism in drug discovery are lacking and there are currently no specific guidelines from regulatory agencies on how to handle this time-dependent chirality despite its prevalence and impact on the pharmaceutical industry.

The present invention exploits the previously unrecognized form of atropisomerism in 3, 4,5-trisubstituted isoxazoles to yield a new class of Nav1.7 inhibitors. This provides opportunities for multiple discoveries with respect to the synthetic design of atropisomeric isoxazoles, their characterization and kinetics of chirality, and the subsequent understanding of how this chirality element affects Nav1.7 potency and selectivity over Nav1.5. More broadly, given the prevalence of substituted isoxazoles in numerous drug discovery programs, which will aid in designing new strategies to recognize and understand the positive or negative impact of atropisomerism across multiple therapeutic programs.

The 3,4,5-trisubstituted isoxazole in accordance with the present invention is based upon general formula I, including a stereoisomer, enantiomer, atropisomer, mixture of enantiomers, mixture of diastereomers, mixture of atropisomers, or isotopic variant thereof; or a pharmaceutically acceptable salts, solvates, hydrates, or prodrugs thereof:

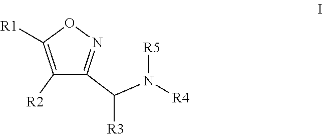

Wherein R1 is selected from the group of methyl, phenyl, chlorophenyl, dichlorophenyl, fluorophenyl, trifluoromethyl, methoxypehnyl, cyanophenyl, pyridine, furan, and thiophene, and combinations thereof;

Wherein R2 is selected from the group of hydrogen, methyl, trifluoromethyl, halogen, alkynyl, phenyl, amide, methylphenyl, and fluoromethylphenyl, and combinations thereof; Wherein R3 is selected from the group of hydrogen, keto, thioketo, and combinations thereof; Wherein R4 is selected from the group of 1-10 carbon branched or straight chain alkyl, hydroxyalky, cyclic, heterocyclic, sulfide, aldehyde, phenyl, and combinations thereof; and Wherein R5 is selected from the group of hydrogen, methyl, and saturated or unsaturated cycloalkanes having 3-6 carbon atoms.

Specific 3, 4, 5-trisubstituted isoxazole compounds having Nav1.7 inhibitor activity are listed in Table 1, below.

TABLE 1

| No. | Compound |
|---|---|
| 100 | 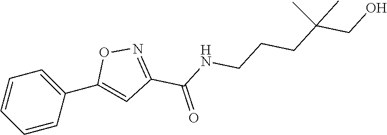 [SMILES: CC(C)(CO)CCCNC(=O)c1cc(-c2ccccc2)on1] |
| 101 | 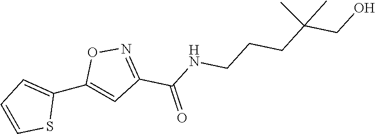 [SMILES: CC(C)(CO)CCCNC(=O)c1cc(-c2cccs2)on1] |
| 102 | 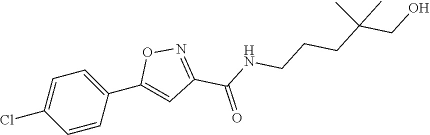 [SMILES: CC(C)(CO)CCCNC(=O)c1cc(-c2ccc(Cl)cc2)on1] |
| 103 | 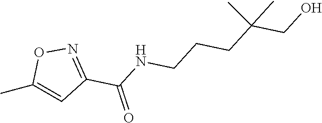 [SMILES: Cc1cc(C(=O)NCCCC(C)(C)CO)no1] |
| 104 | 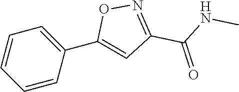 |
| 105 | 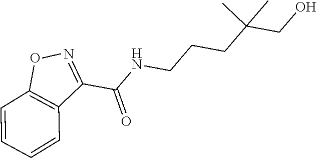 [SMILES: CC(C)(CO)CCCNC(=O)c1noc2ccccc12] |
| 106 | 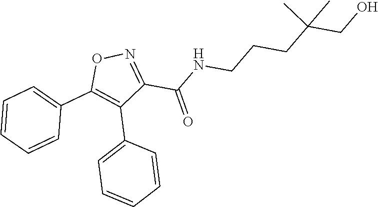 [SMILES: CC(C)(CO)CCCNC(=O)c1noc(-c2ccccc2)c1-c1ccccc1] |
| 107 | 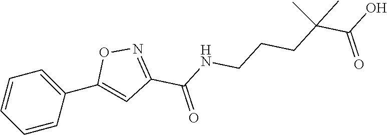 [SMILES: CC(C)(CCCNC(=O)c1cc(-c2ccccc2)on1)C(=O)O] |
| 108 | 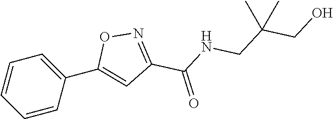 [SMILES: CC(C)(CO)CNC(=O)c1cc(-c2ccccc2)on1] |
| 109 | 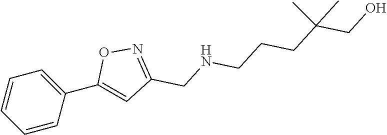 [SMILES: CC(C)(CO)CCCNCc1cc(-c2ccccc2)on1] |
| 110 | 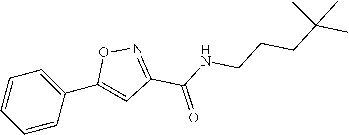 [SMILES: CC(C)(C)CCCNC(=O)c1cc(-c2ccccc2)on1] |
| 111 | 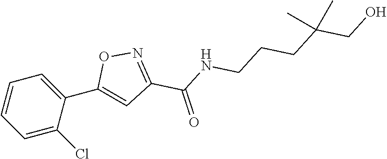 [SMILES: CC(C)(CO)CCCNC(=O)c1cc(-c2ccccc2Cl)on1] |
| 112 | 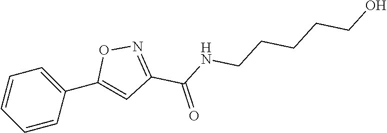 [SMILES: O=C(NCCCCCO)c1cc(-c2ccccc2)on1] |
| 113 | 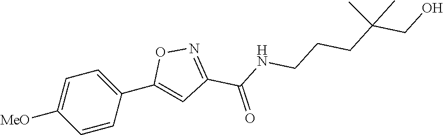 [SMILES: COc1ccc(-c2cc(C(=O)NCCCC(C)(C)CO)no2)cc1] |
| 114 | 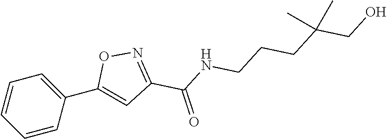 [SMILES: CC(C)(CO)CCCNC(=O)c1cc(-c2ccccc2)on1] |
| 115 | 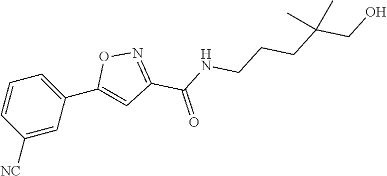 [SMILES: CC(C)(CO)CCCNC(=O)c1cc(-c2cccc(C#N)c2)on1] |
| 116 | 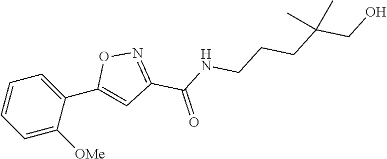 [SMILES: COc1ccccc1-c1cc(C(=O)NCCCC(C)(C)CO)no1] |
| 117 | 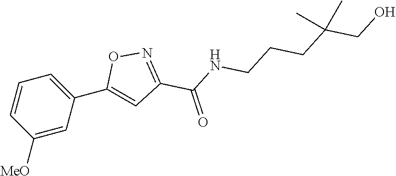 [SMILES: COc1cccc(-c2cc(C(=O)NCCCC(C)(C)CO)no2)c1] |
| 118 | 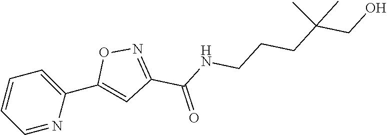 [SMILES: CC(C)(CO)CCCNC(=O)c1cc(-c2ccccn2)on1] |
| 119 | 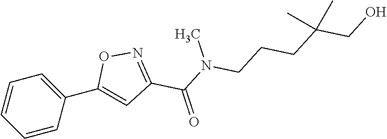 [SMILES: CN(CCCC(C)(C)CO)C(=O)c1cc(-c2ccccc2)on1] |
| 120 | 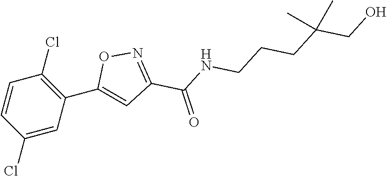 [SMILES: CC(C)(CO)CCCNC(=O)c1cc(-c2cc(Cl)ccc2Cl)on1] |
| 121 | 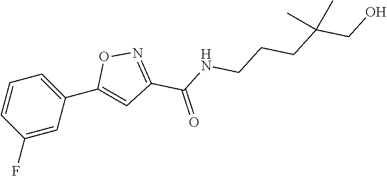 [SMILES: CC(C)(CO)CCCNC(=O)c1cc(-c2cccc(F)c2)on1] |
| 122 | 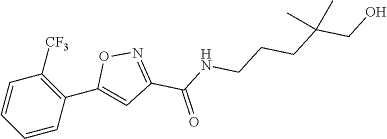 [SMILES: CC(C)(CO)CCCNC(=O)c1cc(-c2ccccc2C(F)(F)F)on1] |
| 123 | 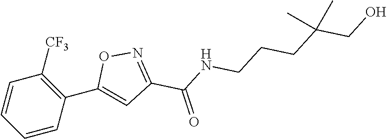 [SMILES: CC(C)(CO)CCCNC(=O)c1cc(-c2ccccc2C(F)(F)F)on1] |
| 124 | 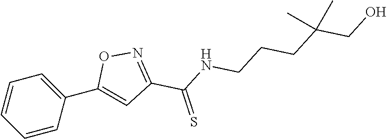 [SMILES: CC(C)(CO)CCCNC(=S)c1cc(-c2ccccc2)on1] |
| 125 | 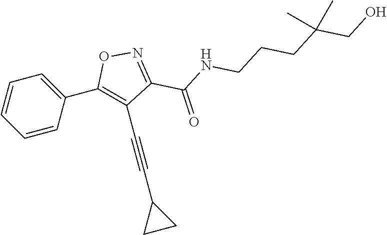 [SMILES: CC(C)(CO)CCCNC(=O)c1noc(-c2ccccc2)c1C#CC1CC1] |
| 126 | 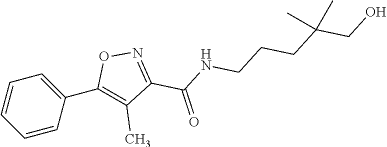 [SMILES: Cc1c(C(=O)NCCCC(C)(C)CO)noc1-c1ccccc1] |
| 127 | 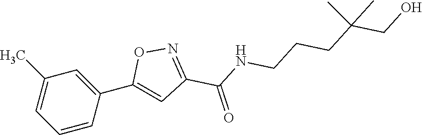 [SMILES: Cc1cccc(-c2cc(C(=O)NCCCC(C)(C)CO)no2)c1] |
| 128 | 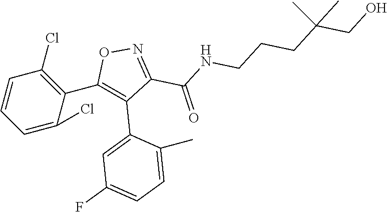 [SMILES: Cc1ccc(F)cc1-c1c(C(=O)NCCCC(C)(C)CO)noc1-c1c(Cl)cccc1Cl] |
| 129 | 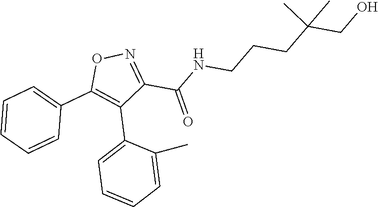 [SMILES: Cc1ccccc1-c1c(C(=O)NCCCC(C)(C)CO)noc1-c1ccccc1] |
| 130 | 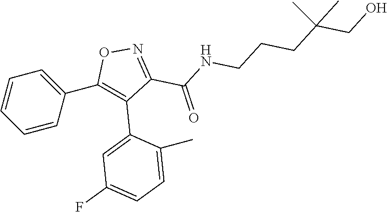 [SMILES: Cc1ccc(F)cc1-c1c(C(=O)NCCCC(C)(C)CO)noc1-c1ccccc1] |
| 131 | 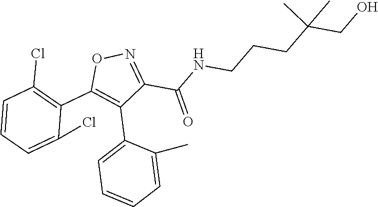 [SMILES: Cc1ccccc1-c1c(C(=O)NCCCC(C)(C)CO)noc1-c1c(Cl)cccc1Cl] |
| 132 | 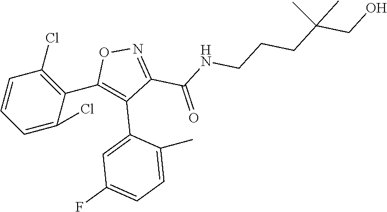 [SMILES: Cc1ccc(F)cc1-c1c(C(=O)NCCCC(C)(C)CO)noc1-c1c(Cl)cccc1Cl] |
| 133 | 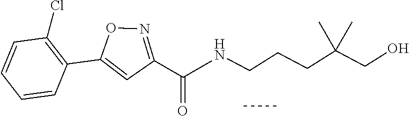 |
| 134 | 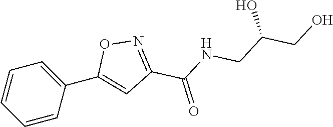 [SMILES: O=C(NC[C@H](O)CO)c1cc(-c2ccccc2)on1] |
| 135 | 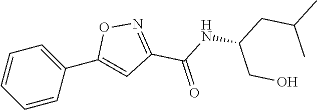 [SMILES: CC(C)C[C@H](CO)NC(=O)c1cc(-c2ccccc2)on1] |
| 136 | 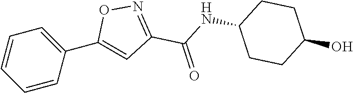 [SMILES: O=C(N[C@H]1CC[C@H](O)CC1)c1cc(-c2ccccc2)on1] |
| 137 | 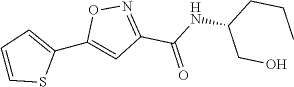 [SMILES: CCC[C@H](CO)NC(=O)c1cc(-c2cccs2)on1] |
| 138 | 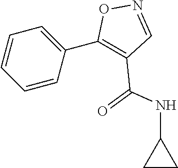 [SMILES: O=C(NC1CC1)c1cnoc1-c1ccccc1] |
| 139 | 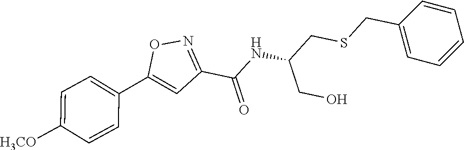 [SMILES: COc1ccc(-c2cc(C(=O)N[C@@H](CO)CSCc3ccccc3)no2)cc1] |
| 140 | 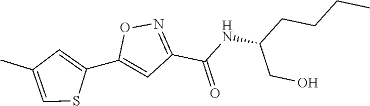 [SMILES: CCCC[C@H](CO)NC(=O)c1cc(-c2cc(C)cs2)on1] |
| 141 | 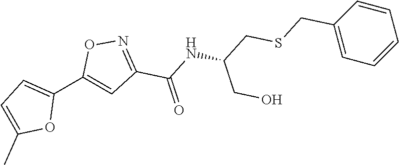 [SMILES: Cc1ccc(-c2cc(C(=O)N[C@@H](CO)CSCc3ccccc3)no2)o1] |
| 142 | 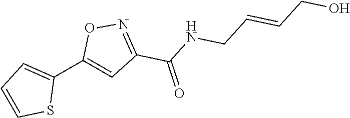 [SMILES: O=C(NC/C=C/CO)c1cc(-c2cccs2)on1] |
| 143 | 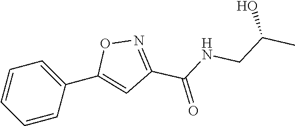 [SMILES: C[C@@H](O)CNC(=O)c1cc(-c2ccccc2)on1] |
| 144 | 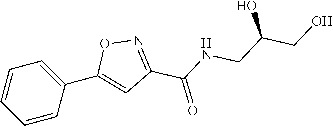 [SMILES: O=C(NC[C@@H](O)CO)c1cc(-c2ccccc2)on1] |
| 145 | 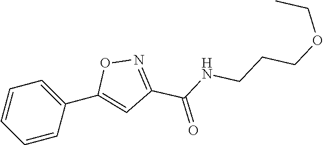 [SMILES: CCOCCCNC(=O)c1cc(-c2ccccc2)on1] |
| 146 | 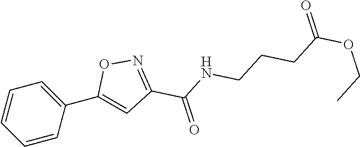 [SMILES: CCOC(=O)CCCNC(=O)c1cc(-c2ccccc2)on1] |
| 147 | 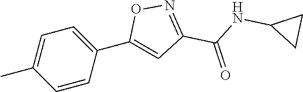 [SMILES: Cc1ccc(-c2cc(C(=O)NC3CC3)no2)cc1] |
| 148 | 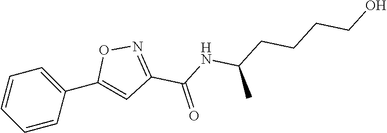 [SMILES: C[C@H](CCCCO)NC(=O)c1cc(-c2ccccc2)on1] |
| 149 | 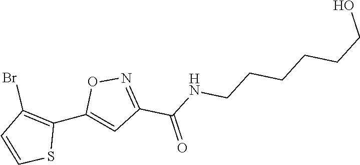 [SMILES: O=C(NCCCCCCO)c1cc(-c2sccc2Br)on1] |
| 150 | 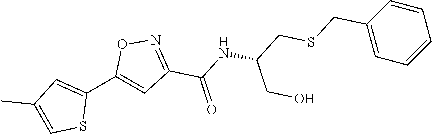 [SMILES: Cc1csc(-c2cc(C(=O)N[C@@H](CO)CSCc3ccccc3)no2)c1] |
| 151 | 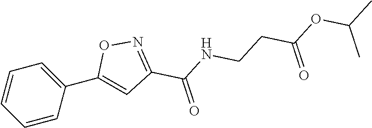 [SMILES: CC(C)OC(=O)CCNC(=O)c1cc(-c2ccccc2)on1] |
| 152 | 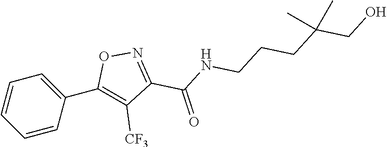 [SMILES: CC(C)(CO)CCCNC(=O)c1noc(-c2ccccc2)c1C(F)(F)F] |

As noted above, SAR studies have demonstrated that methylation of the amide nitrogen or ortho-substitution on the phenyl ring in the 5-position enhances sodium gate channel subtype selectivity (Nav1.7 vs. Nav1.5). Moreover, as is apparent from the enumerated 3,4,5-trisubstituted isoxazole compound structures 100-129 in Table 1, above, subtle structural differences have a profound influence on both IC50 potency and selectivity of Nav1.7 over Nav1.5. In particular, a 4-phenyl substitution increases Nav1.7 inhibition, particularly, where the 4-phenyl substituent is substantially orthogonal to the isoxazole ring. Further, atropisomerism around the carbon-carbon bond at the 4-position affords chirality that enhances Nav1.7 inhibition and selectivity over Nav1.5.

Compound 100, identified through high-throughput screening (HTS), exhibited potency against Nav1.7 (570 nM) but poor selectivity over Nav1.5 (1.9 uM) in in vitro testing. Nonetheless, compound 100 showed promising compound profiling in ADME assays (solubility, CYP inhibition, in vitro metabolism). When compound 100 was tested in in vivo mouse automated formalin model studies, it caused a 31.4% reduction in late-phase pain-induced reactions albeit at a relatively high dose (135 mg/kg p.o.). Subsequent systematic SAR studies demonstrated that subtype selectivity (Nav1.7 vs. Nav1.5) could be improved with methylation of the amide nitrogen, as shown in compound 119 or ortho-substitution on the phenyl ring in the 5-position as in compound 130.

Compound 124 includes a meta-substitution on the phenyl ring in the 5-position that results in reversing subtype selectivity. Compound 124 was found to be very potent against Nav 1.5 (36 nM) as well as Nav1.7 (120 nM) again, supporting that subtle structural differences have a profound influence on both potency and selectivity. Similarly, a significant increase in Nav1.7 potency was realized when a substitution was introduced at the 4-position of the isoxazole ring as exemplified by the 4-phenyl substitution in compound 106. Installation of a 4-phenyl substituent increased Nav1.7 inhibition by almost an order of magnitude (60 nM) over compound 100, yet subtype selectivity remained poor (Nav1.5 IC50=180 nM).

At this point we also became intrigued with the structure of 106. We hypothesized that phenyl substitution at the 4-position would induce a conformational bias to avoid steric interactions between the adjacent phenyl ring in the 5-position and ester group in the 3-position. Indeed, density function theory (DFT) calculations at the B3LYP/6-31G level of theory identified the lowest energy conformation of a simplified analog of compound 106 that demonstrated a clear preference for the 4-phenyl substituent to be almost completely orthogonal to isoxazole ring, as shown in FIG. 4. The DFT calculations were confirmed by X-ray crystallography of similar tri-substituted isoxazoles where a phenyl ring in the 4-position is orthogonal to the isoxazole ring.

### Example 1

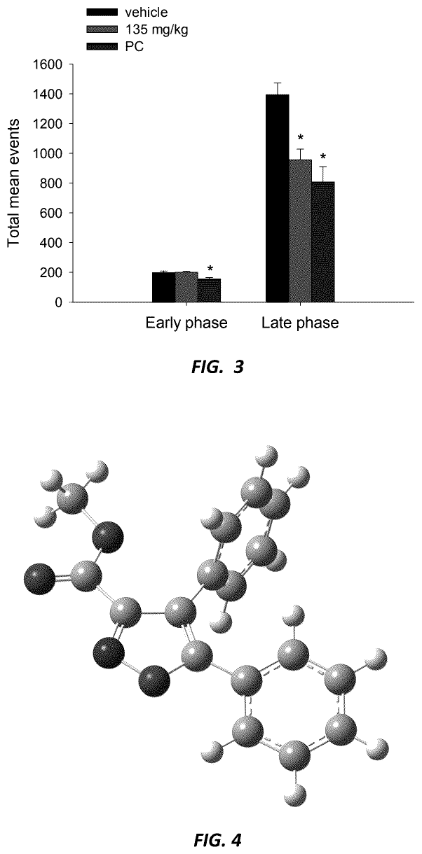

Compound 100 was administered using the mouse automated formalin model at a dosage of 135 mg/kg p.o. Dosage was selected based upon projection from mouse exposure at 5 mg/kg. Early phase and late phase tonic events were measured with a Tmax equal to 15 minutes and a T1/2 equal to 1.1 hours. Table 2, below, and FIG. 3 summarize the results of the test demonstrating the Nav1.7 inhibitory activity of compound 100 relative to vehicle.

TABLE 2

| | | | Mean | | | Mean | | | Percent |
|---|---|---|---|---|---|---|---|---|---|
| Dose | Pre-tx | | plasma, | | | Brain, | | | Reversal in |
| (mg/kg) | Time | h1.7 | u | Fu, p | Plasma | u | Fu, b | Brain | Late Phase |
| p.o. | (hr) | IC50(nM) | (nM) | predicated | TER | (nM) | predicted | TER | Formalin |
| 135 | 0.25 | 2020 (FI) | 282 | 0.028 | 0.1 (FI) | 414 | 0.034 | 0.2 (FI) | 31.4 ± 5.1 |
| | | 230 (SI) | | | 1.2 (SI) | | | 1.8 (SI) | |
| Control | 1 | | | | | | | | 42.1 ± 7.5 |

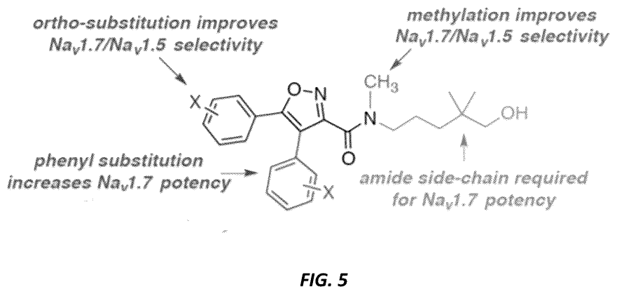

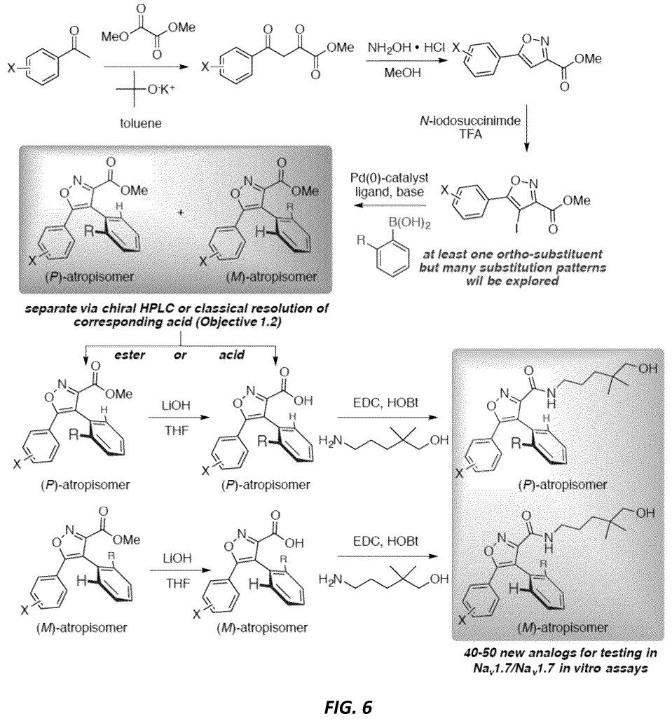

FIG. 5 outlines the synthesis of 3,4,5-trisubstituted isoxazoles in accordance with the present invention. Compound 106 was synthesized by this synthetic route using phenyl boronic acid. Without being bound by theory, atropisomers around the C—C bond at the 4-position are contemplated with either a single ortho- or meta-substituent. Atropisomeric isoxazoles with simple ortho-substitution from aryl boronic acids that includes ortho-fluorine, -chlorine, -bromine, -methyl, and -methoxy may also be made by this synthetic route.

Chiral separation of trisubstituted atropisomeric isoxazoles may be accomplished by interrupting the synthetic route after the Suzuki cross-coupling reaction to yield two complementary methods for separating the atropisomers as shown in FIG. 5. The methyl ester offers an ideal site for chiral semi-preparative HPLC separation. Alternatively, hydrolysis of the methyl ester to the corresponding carboxylic acid as a handle for separation via classical resolution.

1. A sodium gated channel Nav1.7 inhibitor, comprising the compound having the formula I, a stereoisomer, enantiomer, atropisomer, mixture of enantiomers, mixture of diastereomers, mixture of atropisomers, or isotopic variant thereof; or pharmaceutically acceptable salts, solvates, hydrates, or prodrugs thereof: 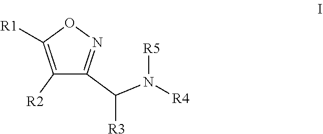
Wherein R1 is selected from the group of methyl, phenyl, chlorophenyl, dichlorophenyl, fluorophenyl, trifluoromethyl, methoxypehnyl, cyanophenyl, pyridine, furan, and thiophene, and combinations thereof;
Wherein R2 is selected from the group of hydrogen, methyl, trifluoromethyl, halogen, alkynyl, phenyl, amide, methylphenyl, and fluoromethylphenyl, and combinations thereof;
Wherein R3 is selected from the group of hydrogen, keto, thioketo, and combinations thereof;
Wherein R4 is selected from the group of 1-10 carbon branched or straight chain alkyl, hydroxyalky, cyclic, heterocyclic, sulfide, aldehyde, phenyl, and combinations thereof; and
Wherein R5 is selected from the group of hydrogen, methyl, and saturated or unsaturated cycloalkanes having 3-6 carbon atoms.

2. The sodium gated channel Nav1.7 inhibitor of claim 1, wherein the compound further comprises:

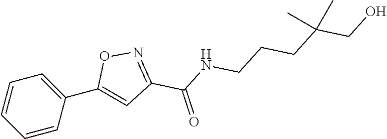 [SMILES: CC(C)(CO)CCCNC(=O)c1cc(-c2ccccc2)on1]

3. The sodium gated channel Nav1.7 inhibitor of claim 1, wherein the compound further comprises:

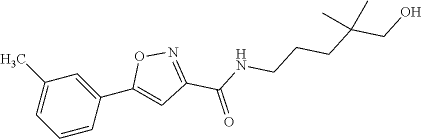 [SMILES: Cc1cccc(-c2cc(C(=O)NCCCC(C)(C)CO)no2)c1]

4. The sodium gated channel Nav1.7 inhibitor of claim 1, wherein the compound further comprises

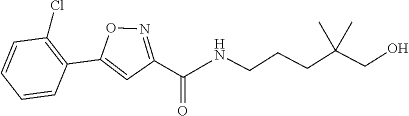

5. The sodium gated channel Nav1.7 inhibitor of claim 1, wherein the compound further comprises:

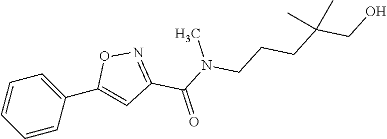 [SMILES: CN(CCCC(C)(C)CO)C(=O)c1cc(-c2ccccc2)on1]

6. The sodium gated channel Nav1.7 inhibitor of claim 1, wherein the compound exhibits Nav1.7 selectivity that is at least two times greater than its selectivity for Nav1.5.

7. The sodium gated channel Nav1.7 inhibitor of claim 1, wherein the compound exhibits Nav1.7 selectivity that is at least three times greater than its selectivity for Nav1.5.

8. The sodium gated channel Nav1.7 inhibitor of claim 1, wherein the R2 substituent group exhibits atropisomerism relative to the R1 substituent group.

9. The sodium gated channel Nav1.7 inhibitor of claim 1, wherein R1 is a phenyl group.

10. The sodium gated channel Nav1.7 inhibitor of claim 9, wherein the phenyl group further has at least one substituent group selected from the group of methyl, methoxy, cyano, chloro, dichloro, fluoro and trifluoromethyl.

11. The sodium gated channel Nav1.7 inhibitor of claim 10, wherein the at least one substituent group is present at one or both of the ortho or meta positions of the phenyl group.

12. A method of inhibiting Nav1.7, comprising the step of administering to a patient in need thereof a pharmaceutically acceptable amount of a compound having the formula I, a stereoisomer, enantiomer, atropisomer, mixture of enantiomers, mixture of diastereomers, mixture of atropisomers, or isotopic variant thereof; or pharmaceutically acceptable salts, solvates, hydrates, or prodrugs thereof: 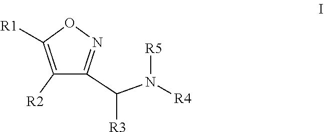
Wherein R1 is selected from the group of methyl, phenyl, chlorophenyl, dichlorophenyl, fluorophenyl, trifluoromethyl, methoxypehnyl, cyanophenyl, pyridine, furan, and thiophene, and combinations thereof;
Wherein R2 is selected from the group of hydrogen, methyl, trifluoromethyl, halogen, alkynyl, phenyl, amide, methylphenyl, and fluoromethylphenyl, and combinations thereof;
Wherein R3 is selected from the group of hydrogen, keto, thioketo, and combinations thereof;
Wherein R4 is selected from the group of 1-10 carbon branched or straight chain alkyl, hydroxyalky, cyclic, heterocyclic, sulfide, aldehyde, phenyl, and combinations thereof; and
Wherein R5 is selected from the group of hydrogen, methyl, and saturated or unsaturated cycloalkanes having 3-6 carbon atoms.

13. The method of claim 12, wherein the administered compound further comprises:

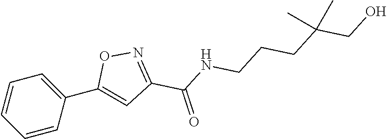 [SMILES: CC(C)(CO)CCCNC(=O)c1cc(-c2ccccc2)on1]

14. The method of claim 12, wherein the administered compound further comprises:

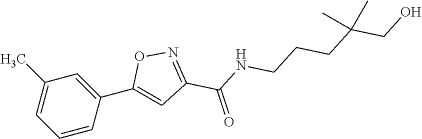 [SMILES: Cc1cccc(-c2cc(C(=O)NCCCC(C)(C)CO)no2)c1]

15. The method of claim 12, wherein the administered compound further comprises:

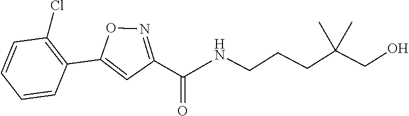

16. The method of claim 12, wherein the administered compound further comprises:

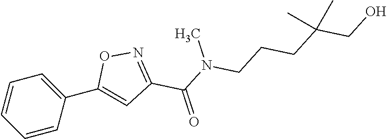 [SMILES: CN(CCCC(C)(C)CO)C(=O)c1cc(-c2ccccc2)on1]

17. The method of claim 12, wherein the administered compound exhibits Nav1.7 selectivity that is at least two times greater than its selectivity for Nav1.5.

18. The method of claim 12, wherein the administered compound exhibits Nav1.7 selectivity that is at least three times greater than its selectivity for Nav1.5.

19. The method of claim 12, wherein the R2 substituent group in the adminstered compound exhibits atropisomerism relative to the R1 substituent group.

20. The method of claim 12, wherein R1 is a phenyl group.

21. The method of claim 20, wherein, wherein the phenyl group further has at least one substituent group selected from the group of methyl, methoxy, cyano, chloro, dichloro, fluoro and trifluoromethyl.

22. The method of claim 21, wherein the at least one substituent group is present at one or both of the ortho or meta positions of the phenyl group.
